(19) United States
(12) Patent Application Publication
(10) Pub. No.: US 2004/0072745 A1
(43) Pub. Date: Apr. 15, 2004

(54) Therapeutic methods of use for lp229

(76) Inventors: Josef Georg Heuer, Indianapolis, IN (US); Kristine Kay Kikly, Fortville, IN (US); Eric Wen Su, Carmel, IN (US)

Correspondence Address: Paula K Davis, Eli Lilly & Company, Patent Division, PO Box 6288, Indianapolis, IN, 46206-6288, US

(21) Appl. No.: 10/250,959

(22) PCT Filed: Jan. 9, 2002
(86) PCT No.: PCT/US02/00487

(51) Int. Cl. A61K 38/17

(52) U.S. Cl. 514/12

(57) ABSTRACT

Methods of use are provided for the treatment or prevention of poor healing or chronic wounds, skin diseases, sepsis, inflammation, immunodeficiencies, autoimmune diseases, infectious diseases, allergic diseases, and malignancies, particularly those disorders associated with the skin, by administering an LP229 polypeptide, protein, or epitope-recognizing antibody thereof to a patient in need of such therapy.

### FIELD OF THE INVENTION

This application claims priority of Provisional Applications Serial No. 60/262,458 filed Jan. 18, 2001, and Serial No. 60/325,233 filed Sep. 27, 2001.

The present invention relates to novel methods of use for LP229 polynucleotides, LP229 polypeptides, or LP229 epitope-recognizing antibodies. The invention provides methods of use in the treatment or prevention of poor healing or chronic wounds, skin diseases, sepsis, inflammation, immunodeficiencies, autoimmune diseases, infectious diseases, allergic diseases, and malignancies, particularly those disorders associated with the skin, and conditions or symptoms related thereto by administering an LP229 polynucleotide, polypeptide, or antibody to a patient in need of such therapy.

### BACKGROUND OF THE INVENTION

Wound healing is a complex biological process involving extracellular matrix, blood cells, parenchymal cells, and mediators such as cytokines. After the wound reaches hemostasis, the point where bleeding stops, the healing process begins. It occurs in three stages: inflammation, tissue formation (proliferation), and tissue regeneration (remodeling). Healing begins very quickly after injury occurs; for example, re-epithelialization of cutaneous wounds begins within hours (Singer and Clark, New Eng. J. Med. 341(10): 738-46 (1999)). However, chronic wounds, such as pressure ulcers or burns, may take months or even years to heal completely.

Healing is regulated by growth factors and cytokines that affect cell migration, proliferation, and protein production. In hemostasis, proteins such as fibrin and fibronectin interact to clot the blood, and cytokines and growth factors are upregulated. Within four to six hours after injury, inflammation begins. During inflammation, neutrophils (polymorphonuclear leukocytes, PMNS), monocytes and macrophages infiltrate the wound. These phagocytic cells release growth factors for the proliferative phase, enzymatic mediators (proteases) that degrade proteins, and phagocytose bacteria, dead and dying cells thus debriding the wound.

In the next phase, proliferation begins. Collagen is deposited, forming scar tissue. Fibroblasts produce proteoglycans, which bind the collagen fibers together. Over time, the collagen is degraded by proteases and remodeled into a stronger scar structure.

Although they are necessary for proper function during the phases of inflammation and remodeling, proteases can cause serious tissue damage if improperly regulated. Serine proteases such as elastase and cathepsin G degrade collagen, proteoglycans, and elastin, a protein found in walls of arteries (Gleisner, “Lysosomal factors in inflammation,” Immunology of Inflammation, Peter Ward, Ed., Elsevier, N.Y. 1983). Additionally, these proteases contribute to activation of matrix metalloproteinases, which cause further destruction of tissue (Zhang, et al., J. Clin. Invest. 99(5): 894-900 (1997)). This degradation can lead to prolonged wound healing. In fact, studies have shown that many chronic wounds contain increased levels of degradative serine proteases. In a normal wound, these proteases are inhibited by naturally occurring protease inhibitors that bind the protease, thus inactivating it. But in the absence of inhibitors, these proteases are not regulated, yielding increased degradation of wound healing proteins.

Furthermore, serine proteases are associated with numerous other chronic inflammatory disease states. In inflamed joints, serine proteases lead to the destruction of collagen and proteoglycans of articular cartilage associated with arthritis (Song, et al., J. Exp. Med. 190(4): 535-42 (1999)). Serine proteases degrade fibrinogen and fibronectin, proteins involved with homeostasis (U.S. Pat. No. 6,262,020). Loss of homeostasis can lead to uncontrolled coagulation, fibrinolysis, and inflammation, resulting in sepsis. Serine proteases aid in the spread of the human immunodeficiency virus (HIV). Inside the nucleus of an infected cell, proteases cleave HIV proteins into active peptides, necessary for proper replication. In graft-versus-host disease (GVHD), serine proteases may be involved in the cytotoxic mechanism following bone marrow transplantation (Maiocchi, et al., Haematologica 83(8): 686-9 (1998)).

Secretory leukocyte protease inhibitor (SLPI) is a serine protease inhibitor. It has been shown to inhibit proteases such as elastase and cathepsin G, indicating its usefulness in treating diseases associated with serine protease activity. Additionally, SLPI is known to inhibit NFκB, thereby modulating cytokine and chemokine activity (Ashcroft, et al., Nat. Med. 6(10): 1147-53 (2000)). Therapeutic uses for SLPI include treatment of chronic wounds (Ashcroft, supra), HIV (Baqui, et al., Clin. Diagn. Lab. Immunol. 6(6): 808-11 (1999)), degenerative and inflammatory arthritis (Song, et al., J. Exp. Med. 190(4): 535-42 (1999)), and hepatic ischemia and reperfusion (Lentsch, et al., Gastroenterology 117(4): 953-61 (1999)). Moreover, SLPI exhibits antibacterial, antifungal, and antiviral activities, and has been shown to antagonize lipopolysaccharide (LPS)-induced pro-inflammatory mediator synthesis (Ashcroft, supra).

SLPI is a 12 kDa protein composed of two cysteine-rich domains with a protease inhibitory region site at leucine 72 in the carboxy-terminal domain (Ashcroft, et al., Nat. Med. 6(10): 1147-53 (2000)). A comparison of the sequence similarity between known SLPI family members and LP229 is shown in Table 1 (Song, et al., J. Exp. Med. 190(4): 535-42 (1999)).

TABLE 1
Comparison of SLPI family members.

| | | |
|---|---|---|
| mSLPI | MKSCGLLPFTVLLALGILAPWTVEGGKNDAIKIGACPAKKPAQCLKLEKP | 50 |
| rSLPI | MKSCGLFPLMVLLALGVLAPWTVEGGKNDAIKIGACPAKKPAQCLKLEKP | 50 |
| hSLPI | MKSSGLFPFLVLLALGTLAPWAVE•GSGKSFKAGVCPPKKSAQCLRYKKP | 49 |
| LP229 | MRTQSLLLLGALLAVGSQLP•AVF•GRKKGEKSGGCPP•DDGPCLLSVPD | 47 |
| mSLPI | QCRTDWECPGKQRCCQDACGSKCVNPVPIRKPVWRKPGRCVKTQARCMML | 100 |
| rSLPI | ECGTDWECPGKQRCCQDTCGFKCVNPVPIRGPVKKKPGRCVKFQGKCLML | 100 |
| hSLPI | ECQSDWQCPGKKRCCPDTCGIKCLDPVDTPNPTRRKPGKCPVTYGQCLML | 99 |
| LP229 | QCVEDSQCPLTRKCCYRACFRQCVPRVSV••••••KLGSCPEDQLRCL•• | 89 |
| mSLPI | NPPN•VCQRDGQCDGKYKCCEGICGKVCLPPM˜˜ | 131 |
| rSLPI | NPPN•KCQNDGQCDGKYKCCEGMCGKVCLPPV˜˜ | 131 |
| hSLPI | NPPN•FCEMDGQCKRDLKCCMGMCGKSCVSPVKA | 132 |
| LP229 | SPMNHLCYKDSDCSGKKRCCHSACGRDCRDPARG | 123 |

Because of its biological activity, SLPI is useful for the treatment of chronic wounds, infections, inflammation and diseases related to inflammation. Discovery of new molecules with similar biological activity and therapeutic activity is sought. Molecules which share sequence similarity with SLPI may exhibit these activities.

### BRIEF SUMMARY OF THE INVENTION

The present invention concerns methods for treating or preventing poor healing or chronic wounds, skin diseases, sepsis, inflammation, immunodeficiencies, autoimmune diseases, infectious diseases, allergic diseases, and malignancies, particularly those disorders associated with the skin, and conditions or symptoms related thereto, by administering LP229 polypeptides. This invention also provides methods for treating or preventing skin diseases, sepsis, inflammation, immunodeficiencies, autoimmune diseases, infectious diseases, allergic diseases, and malignancies, particularly those disorders associated with the skin, by administering LP229 antagonists including antibodies or soluble receptors.

It is a further objective of the invention to provide methods and means for intervening in the underlying mechanisms of poor healing or chronic wounds, skin diseases, sepsis, inflammation, immunodeficiencies, autoimmune diseases, infectious diseases, allergic diseases, and malignancies, particularly those disorders associated with the skin, by administering LP229 polypeptides or antibodies to a patient in need of such intervention. Such methods and means expressly include methods for intervening by inhibiting the action of agents that cause or mediate conditions and symptoms of poor healing or chronic wounds, skin diseases, sepsis, inflammation, immunodeficiencies, autoimmune diseases, infectious diseases, allergic diseases, and malignancies, particularly those disorders associated with the skin, by administration of LP229 polypeptides or LP229 epitope-recognizing antibodies.

Preferred polynucleotides for practicing the present invention are those that encode the full-length LP229 polypeptide as shown in SEQ ID NO:2. Similarly, preferred polypeptides for practicing the present invention are the full-length LP229 polypeptide as shown in SEQ ID NO:2.

### DETAILED DESCRIPTION OF THE INVENTION

In one embodiment, novel utility is contemplated for LP229 polypeptides comprising the amino acid sequence of the open reading frame encoded by the polynucleotide sequence as shown in SEQ ID NO:1. The isolated nucleic acid comprises DNA consisting of nucleotides 90 or about 162 through about 461, inclusive, of SEQ ID NO:1.

TABLE 2
LP229 polynucleotide, SEQ ID NO:1.

| | |
|---|---|
| CTTTCCTCTC CTGACTAAGT TTCTCTGGCT TCCCTGAGGC TGCAGGTGTT AATCTGGGGG | 60 |
| GCCCTGGGCC CTGAGCCGGC AGCAGAAAT ATG AGG ACC CAG AGC CTT CTC CTC | 143 |
| Met Arg Thr Gln Ser Leu Leu Leu | |
| 1 5 | |
| CTG GGG GCC CTC CTG GCT GTG GGG AGT CAG CTG CCT GCT GTC TTT GGC | 191 |
| Leu Gly Ala Leu Leu Ala Val Gly Ser Gln Leu Pro Ala Val Phe Gly | |
| 10 15 20 | |
| AGG AAG AAG GGA GAG AAA TCG GGG GGC TGC CCG CCA GAT GAT GGG CCC | 239 |
| Arg Lys Lys Gly Glu Lys Ser Gly Gly Cys Pro Pro Asp Asp Gly Pro | |
| 25 30 35 40 | |
| TGC CTC CTA TCG GTG CCT GAC CAG TGC GTG GAA GAC AGC CAG TGT CCC | 287 |
| Cys Leu Leu Ser Val Pro Asp Gln Cys Val Glu Asp Ser Gln Cys Pro | |
| 45 50 55 | |
| TTG ACC AGG AAG TGC TGC TAC AGA GCT TGC TTC CGC CAG TGT GTC CCC | 335 |
| Leu Thr Arg Lys Cys Cys Tyr Arg Ala Cys Phe Arg Gln Cys Val Pro | |
| 60 65 70 | |
| AGG GTC TCT GTG AAG CTG GGC AGC TGC CCA GAG GAC CAA CTG CGC TGC | 383 |
| Arg Val Ser Val Lys Leu Gly Ser Cys Pro Glu Asp Gln Leu Arg Cys | |
| 75 80 85 | |
| CTC AGC CCC ATG AAC CAC CTG TGT TAC AAG GAC TCA GAC TGC TCG GGC | 431 |
| Leu Ser Pro Met Asn His Leu Cys Tyr Lys Asp Ser Asp Cys Ser Gly | |
| 90 95 100 | |
| AAA AAG CGA TGC TGC CAC AGC GCC TGC GGG CGG GAT TGC CGG GAT CCT | 479 |
| Lys Lys Arg Cys Cys His Ser Ala Cys Gly Arg Asp Cys Arg Asp Pro | |
| 105 110 115 120 | |
| GCC AGA GGC TAA TTCTGATTTA GGATCTGTGG CTCTGCACCT AAGCTGGGGA | 531 |
| Ala Arg Gly | |
| 123 | |
| CCAACGGAAA GAGTTCACGA TGGGAGGCCT GGGGCCCTGC CCGCTGGACA GCACTATCTC | 591 |
| TACCAGCGGT GGTTCCAGCC TTCTGATAAT CACTGGCCTG CTGACACTTC CCTGCAACCC | 651 |
| ATCCACCCCT GGTTTCTCCT CCTGGGAGTC AAAGTCCATA GCCTGAGCTC GGAGGAAGGC | 711 |
| CTCTGTATCA CCCCAGTACT CTGCACCACT GCCATACGAG CTTCCCACCC TTCCTAACGC | 771 |
| TTTCACACCA ATCCGTACAT GCTGCTTCCT CCACCAAAAA TGCCCAATTC AGGCAGACCC | 831 |
| TGACCTCTCC CTCAGGCAGC CCAACCATCC AGAATGAATA TTCTTGCAGA GTTTTCCAAA | 891 |
| CATCAGTCAT TCACCTCTTT CATGATTTTC ACCATACCTA CAAAATAGCA CCATGATAGG | 951 |
| TTGCACGCTG CCTGTACCAC CATTTACTTA ATGTTTTCTT TAAATGGCTC ACTTTTGTAT | 1011 |
| ATAAATAAAT TCATTTCAAA AAAAAAAAAA AAGGGCGGCC GCCGACTAGT GAGCTCGTCG | 1071 |

In one embodiment, the invention provides novel utility for isolated nucleic acid molecules comprising DNA encoding LP229 polypeptides. In another embodiment, the invention provides novel utility for isolated nucleic acid molecules comprising DNA that encodes LP229 polypeptides having amino acid residues from 1 or about 25 to about 123, inclusive, of SEQ ID NO:2, or that are complementary to such encoding nucleic acid sequences, and remain stably bound to them under at least moderate, and optionally, high stringency conditions. Specifically, polypeptides used in the present invention comprise the amino acid sequence as shown in SEQ ID NO:2, as well as fragments, variants, and derivatives thereof.

TABLE 3
LP229 Polypeptide, SEQ ID NO:2.

```
Met Arg Thr Gln Ser Leu Leu Leu Leu Gly Ala Leu Leu Ala Val Gly
    1 5 10 15
Ser Gln Leu Pro Ala Val Phe Gly Arg Lys Lys Gly Glu Lys Ser Gly
                          20 25 30
Gly Cys Pro Pro Asp Asp Gly Pro Cys Leu Leu Ser Val Pro Asp Gln
                  35 40 45
Cys Val Glu Asp Ser Gln Cys Pro Leu Thr Arg Lys Cys Cys Tyr Arg
          50 55 60
Ala Cys Phe Arg Gln Cys Val Pro Arg Val Ser Val Lys Leu Gly Ser
  65 70 75 80
Cys Pro Glu Asp Gln Leu Arg Cys Leu Ser Pro Met Asn His Leu Cys
                                  85 90 95
Tyr Lys Asp Ser Asp Cys Ser Gly Lys Lys Arg Cys Cys His Ser Ala
                        100 105 110
Cys Gly Arg Asp Cys Arg Asp Pro Ala Arg Gly
                115 120 123
```

LP229 has sequence similarity to the secretory leukocyte protease inhibitor (SLPI) family of proteins. (See Table 1, supra.) Like other family members, it contains two regions containing numerous conserved cysteines, an N-terminal region, and a C-terminal region. The C-terminal region also contains a conserved leucine, which may contribute to the serine protease inhibitory effect of this family of proteins. Accordingly, compositions comprising LP229 polypeptides or polynucleotides are useful for the diagnosis, treatment, and intervention of poor healing and chronic wounds such as pressure ulcers, diabetic ulcers, venous stasis ulcers, burns, and other wounds to tissues such as the gastrointestinal tract. In another aspect, compositions comprising LP229 polypeptides or polynucleotides are useful for the diagnosis, treatment, and intervention of sepsis, gram-negative bacteremia, gram-positive bacteremia, inflammation, bacterial, viral and fungal infections, and autoimmune diseases and related conditions caused by the human immunodeficiency virus type 1 (HIV-1). Compositions comprising LP229 epitope-recognizing antibodies or antagonists are useful for the diagnosis, treatment, and intervention of skin diseases, sepsis, inflammation, immunodeficiencies, autoimmune diseases, infectious diseases, allergic diseases, and malignancies, particularly those disorders associated with the skin.

Definitions

The following definitions of terms are intended to correspond to those as well known in the art. The following terms are therefore not limited to the definitions given but are used according to the state of the art, as demonstrated by cited and/or contemporary publications or patents.

“Active” or “activity” for the purposes herein refers to forms of LP229 that retain the biologic and/or immunologic activities of LP229 polypeptide. Elaborating further, “biological” activity refers to a biological function (either inhibitory or stimulatory) caused by a native or naturally occurring LP229 polypeptide other than the ability to induce the production of an antibody against an antigenic epitope possessed by a native or naturally-occurring LP229 polypeptide. An “immunological” activity refers only to the ability to induce the production of an antibody against an antigenic epitope possessed by a native or naturally occurring LP229 polypeptide. A preferred biological activity includes, for example, the ability to treat poor healing or chronic wounds, skin diseases, sepsis, inflammation, immunodeficiencies, autoimmune diseases, infectious diseases, allergic diseases, and malignancies, particularly those disorders associated with the skin.

The term “amino acid” is used herein in its broadest sense and includes naturally occurring amino acids as well as non-naturally-occurring amino acids, including amino acid analogs and derivatives. The latter includes molecules containing an amino acid moiety. One skilled in the art will recognize, in view of this broad definition, that reference herein to an amino acid includes, for example, naturally-occurring proteogenic L-amino acids; D-amino acids; chemically modified amino acids, such as amino acid analogs and derivatives; naturally-occurring non-proteogenic amino acids such as norleucine, beta-alanine, ornithine, etc.; and chemically synthesized compounds having properties known in the art to be characteristic of amino acids. As used herein, the term “proteogenic” indicates that the amino acid can be incorporated into a peptide, polypeptide, or protein in a cell through a metabolic pathway.

The term “antagonist” is used in the broadest sense and includes any molecule that partially or fully blocks, inhibits, or neutralizes a biological activity of a native LP229 polypeptide disclosed herein. In a similar manner, the term “agonist” is used in the broadest sense and includes any molecule that mimics a biological activity of a native LP229 polypeptide disclosed herein. Suitable agonist or antagonist molecules specifically include agonist or antagonist antibodies or antibody fragments, fragments or amino acid sequence variants of native polypeptides, peptides, ribozymes, antisense nucleic acids, small organic molecules, etc. Methods for identifying agonists or antagonists of an LP229 polypeptide may comprise contacting an LP229 polypeptide with a candidate agonist or antagonist molecule and measuring a detectable change in one or more biological activities normally associated with the LP229 polypeptide.

“Antibodies” (Abs) and “immunoglobulins” (Igs) are glycoproteins having the same structural characteristics. Antibodies exhibit binding specificity to a specific antigen. The term “antibody” is used in the broadest sense and specifically covers, without limitation, intact monoclonal antibodies (MAbs), polyclonal antibodies, modified antibodies as known in the art (e.g., chimeric, humanized, recombinant, resurfaced, or CDR-grafted), anti-idiotypic (anti-id) antibodies, and antibody fragments, so long as they exhibit the desired biological activity.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), such as IgG1, IgG2, IgG3, IgG4, IgA and IgA2.

“Antibody fragments” comprise a portion of an intact antibody, preferably the antigen binding or variable region of the intact antibody. Examples of antibody fragments include Fab, Fab′, F(ab′)₂ and Fv fragments; diabodies; linear antibodies (Zapata, et al., Protein Engin. 8 (10): 1057-62 (1995)); single-chain antibody molecules; and multispecific antibodies (e.g., bispecific antibodies) formed from antibody fragments.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight polypeptides (less than about 10 residues); proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONIC®.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption but, rather, is cyclic in nature.

“Conservative substitution” or “conservative amino acid substitution” refers to a replacement of one or more amino acid residue(s) in a protein or peptide. Conservative substitutions of interest are shown in Table 4 along with preferred substitutions. If such substitutions maintain or improve the desired function, then more substantial changes, listed as exemplary substitutions in Table 4, or as further described below in reference to amino acid classes, are introduced and the products screened.

TABLE 4
Conservative Substitutions

| Original | Example | Preferred |
|---|---|---|
| Residue | Substitutions | Substitutions |
| Ala (A) | val, leu, ile | val |
| Arg (R) | lys, gln, asn | lys |
| Asn (N) | gln | gln |
| Asp (D) | glu | glu |
| Cys (C) | ser | ser |
| Gln (Q) | asn | asn |
| Glu (E) | asp | asp |
| Gly (G) | pro, ala | ala |
| His (H) | asn, gln, lys, arg | arg |
| Ile (I) | leu, val, met, ala, phe, | leu |
| | norleucine | |
| Leu (L) | norleucine, ile, val, | ile |
| | met, ala, phe | |
| Lys (K) | arg, gln, asn | arg |
| Met (M) | leu, phe, ile | leu |
| Phe (F) | leu, val, ile, ala, tyr | leu |
| Pro (P) | ala | ala |
| Ser (S) | thr | thr |
| Thr (T) | ser | ser |
| Trp (W) | tyr, phe | tyr |
| Tyr (Y) | trp, phe, thr, ser | phe |
| Val (V) | ile, leu, met, phe, ala, | leu |
| | norleucine | |

Naturally occurring residues are divided into groups based on common side-chain properties:

(1) hydrophobic: cys, ser, thr;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404 097, WO 93/11161, and Holliger, et al., Proc. Natl. Acad. Sci. USA 90(14): 6444-8 (1993).

The term “epitope tagged,” where used herein, refers to a chimeric polypeptide comprising an LP229 polypeptide or domain sequence thereof, fused to a “tag polypeptide”. The tag polypeptide has enough residues to provide an epitope against which an antibody may be made, or which can be identified by some other agent, yet is short enough such that it does not interfere with the activity of the LP229 polypeptide. The tag polypeptide preferably is also fairly unique so that the antibody does not substantially cross-react with other epitopes. Suitable tag polypeptides generally have at least six amino acid residues and usually between about eight to about fifty amino acid residues, preferably, between about ten to about twenty residues.

Papain digestion of antibodies produces two identical antigen-binding fragments, called “Fab” fragments, each with a single antigen-binding site and a residual “Fc” fragment, a designation reflecting the ability to crystallize readily. Papain digestion provides one means of obtaining an immunoglobulin constant domain.

The Fab fragment also contains the constant domain of the light chain and the “first constant domain” (CHI) of the heavy chain. Fab fragments differ from Fv fragments by the addition of a few residues at the carboxy terminus of the heavy chain CHI domain including one or more cystines from the antibody hinge region. Fab′-SH is the designation herein for Fab′ in which the cystine residue(s) of the constant domains bear a free thiol group. F(ab′)₂ antibody fragments were originally produced as pairs of Fab′ fragments which have hinge cystines between them. Other chemical couplings of antibody fragments are also known.

The term “FLAG tag,” where used herein, refers to the LP229 polypeptide sequence fused to a polypeptide sequence containing the amino acid residues DYKDDDDKHV. The FLAG tag is added to one terminus of the polypeptide to aid in purification.

The term “FLIS tag,” where used herein, refers to the LP229 polypeptide sequence fused to a polypeptide sequence containing a FLAG tag plus six histidine residues. The FLIS tag has enough histidine residues to provide a unique purification means to select for the properties of the repeated histidine residues, yet is short enough such that it does not interfere with the activity of the LP229 polypeptide. Suitable tag polypeptides generally have at least six amino acid residues and usually between about four to about twenty amino acid residues, such as the polypeptide sequence GAFIDYKDDDDKHVHHHHHH.

The term “fragment thereof” as used herein refers to a fragment, piece, or sub-region of a nucleic acid or protein molecule whose sequence is disclosed herein, such that the fragment comprises 5, 10, 15, 20 or more amino acids, or 15, 30, 45, 60 or more nucleotides that are contiguous in the parent protein or nucleic acid compound. When referring to a nucleic acid compound, “fragment thereof” refers to 15, 30, 45, 60 or more contiguous nucleotides, derived from the parent nucleic acid, and also, owing to the genetic code, to the complementary sequence. For example, if the fragment entails the sequence 5′-AGCTAG-3′, then “fragment thereof” would also include the complementary sequence, 3′-TCGATC-5′.

“Functional fragment” or “functionally equivalent fragment,” as used herein, refers to a region or fragment of a full-length protein or sequence of amino acids that are capable of competing with the endogenous or native LP229 polypeptide for binding to a natural or recombinantly expressed LP229 polypeptide receptor. The present invention also provides for the use of fragments of the LP229 polypeptides disclosed herein wherein said fragments retain ability to bind a natural ligand. As used herein, “functional fragments” includes fragments, whether or not fused to additional sequences, which retain and exhibit, under appropriate conditions, measurable bioactivity. Functional fragments of the proteins disclosed herein may be produced as described herein, preferably using cloning techniques to engineer smaller versions of the functioning LP229 polypeptide, lacking sequence from the 5′ end, the 3′ end, from both ends, or from an internal site.

Functional analogs of the LP229 protein may be generated by deletion, insertion, or substitution of one or more amino acid residues. The present invention includes methods of using LP229 proteins as well as any related functional analogs that retain the ability to be employed therapeutically according to the present invention. Modifications of the amino acid sequence can generally be made in accordance with the substitutions provided in Table 4.

The term “fusion protein” denotes a hybrid protein molecule not found in nature comprising a translational fusion or enzymatic fusion in which two or more different protein segments not naturally found in a contiguous sequence are covalently linked together, generally on a single peptide chain.

“Fv” is the minimum antibody fragment that contains a complete antigen-recognition and binding site. This region consists of a dimer of one heavy- and one light-chain variable domain in tight, non-covalent association. It is in this configuration that the three CDRs of each variable domain interact to define an antigen-binding site on the surface of the V_{H}-V_{L} diner. Collectively, the six CDRs confer antigen-binding specificity to the antibody. However, even a single variable domain (or half of an Fv comprising only three CDR specific for an antigen) has the ability to recognize and bind antigen, although at a lower affinity than the entire binding site.

The term “homolog” or “homologous” describes the relationship between different nucleic acid compounds or amino acid sequences in which said sequences or molecules are related by partial identity or similarity at one or more blocks or regions within said molecules or sequences.

The term “host cell” as used herein refers to any eukaryotic or prokaryotic cell that is suitable for propagating and/or expressing a cloned gene contained on a vector that is introduced into said host cell by, for example, transformation or transfection, or the like.

The term “hybridization” as used herein refers to a process in which a single-stranded nucleic acid compound joins with a complementary strand through nucleotide base pairing. The degree of hybridization depends upon, for example, the degree of sequence similarity, the stringency of hybridization, and the length of hybridizing strands. “Selective hybridization” refers to hybridization under conditions of high stringency.

As used herein, the term “immunoadhesin,” also referred to as an Fc fusion, designates antibody-like molecules that combine the binding specificity of a heterologous protein (an “adhesin”) with the effector functions of immoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand.

Administration “in combination with” one or more additional therapeutic agents includes simultaneous (concurrent) and consecutive administration in any order.

“Isolated,” when used to describe the various polypeptides, polynucleotides, or antibodies disclosed herein, means a polypeptide, polynucleotide, or antibody that has been identified and separated and/or recovered from a component of its natural environment. Preferably, the isolated polypeptide, polynucleotide, or antibody is free of association with all components with which it is naturally associated. Contaminant components of its natural environment are materials that would typically interfere with diagnostic, prophylactic, or therapeutic uses for the polypeptide, polynucleotide, or antibody and may include enzymes, hormones, and other proteinaceous or non-proteinaceous solutes. In preferred embodiments, the polypeptide, polynucleotide, or antibody will be purified (1) to greater than 95% purity by weight of polypeptide, polynucleotide, or antibody, as determined by the Lowry method, and most preferably more than 99% by weight, (2) to homogeneity by SDS-PAGE under reducing or non-reducing conditions using Coomassie blue, or preferably, silver stain. Isolated polypeptide, polynucleotide, or antibody includes polypeptide, polynucleotide, or antibody in situ within recombinant cells, since at least one component of the LP229 polypeptide, polynucleotide, or antibody's natural environment will not be present. Ordinarily, however, isolated polypeptide, polynucleotide, or antibody will be prepared by at least one purification step.

The “light chains” of antibodies (immunoglobulins) from any vertebrate species can be assigned to one of two clearly distinct types, called kappa and lambda, based on the amino acid sequences of their constant domains.

A “liposome” is a small vesicle composed of various types of lipids, phospholipids and/or surfactant that is useful for delivery of a drug (such as an LP229 polypeptide or antibody thereto) to a mammal. The components of the liposome are commonly arranged in a bilayer formation, similar to the lipid arrangement of biological membranes.

The polynucleotides of the present utility invention are designated herein as “LP229 polynucleotide(s)” or “LP229 polypeptide-encoding polynucleotide(s).” The polypeptides of the present invention are designated herein as “LP229 polypeptide(s)” or “LP229 protein(s).” The term “LP229” refers to a specific group of molecules as defined herein. A complete designation wherein the term “LP229” is immediately followed by a numerical designation plus a molecule type (e.g., LP229 polypeptide or LP229 polynucleotide) refers to a specific type of molecule within the designated group of molecules as defined herein. The LP229 molecules described herein may be isolated from a variety of sources including, but not limited to, human tissue types, or prepared by recombinant or synthetic methods.

The LP229 polynucleotide can be composed of any polyribonucleotide or polydeoxyribonucleotide, which may be unmodified RNA or DNA or modified RNA or DNA. For example, the LP229 polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the LP229 polynucleotides can be composed of triple-stranded regions comprising RNA or DNA or both RNA and DNA. LP229 polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The term “LP229 polypeptide” specifically encompasses truncated or secreted forms of an LP229 polypeptide (e.g., soluble forms containing, for instance, an extracellular domain sequence), variant forms (e.g., alternatively spliced forms), and allelic variants of an LP229 polypeptide.

In one embodiment, the native sequence LP229 polypeptide is a full-length or mature LP229 polypeptide comprising amino acids 1 or about 25 through 123 of SEQ ID NO:2. Also, while the LP229 polypeptides disclosed herein are shown to begin with a methionine residue designated as amino acid position 1, it is conceivable and possible that another methionine residue located either upstream or downstream from amino acid position 1 may be employed as the starting amino acid residue.

LP229 sequences can be isolated from nature or can be produced by recombinant or synthetic means. LP229 polypeptides include, but are not limited to, deglycosylated, unglycosylated, and modified glycosylated forms of LP229 polypeptides, as well as sufficiently homologous forms having conservative substitutions, additions, or deletions of the amino acid sequence, as well as portions thereof such that the molecule retains LP229-like functionality and bioactivity.

The term “LP229 polypeptide(s)” is also meant to encompass polypeptides containing pro-, or prepro-sequences, that when processed result in the production of the LP229 polypeptide.

An “LP229 variant polynucleotide” or “LP229 variant nucleic acid sequence” means an active LP229 polypeptide-encoding nucleic acid molecule as defined below, having at least about 75% nucleic acid sequence identity with SEQ ID NO:1. Ordinarily, an LP229 variant polynucleotide will have at least about 90% nucleic acid sequence identity, yet more preferably at least about 91% nucleic acid sequence identity, yet more preferably at least about 92% nucleic acid sequence identity, yet more preferably at least about 93% nucleic acid sequence identity, yet more preferably at least about 94% nucleic acid sequence identity, yet more preferably at least about 95% nucleic acid sequence identity, yet more preferably at least about 96% nucleic acid sequence identity, yet more preferably at least about 97% nucleic acid sequence identity, yet more preferably at least about 98% nucleic acid sequence identity, yet more preferably at least about 99% nucleic acid sequence identity with the nucleic acid sequences shown in SEQ ID NO:1. Variants specifically exclude or do not encompass the native nucleotide sequence, as well as those prior art sequences that share 100% identity with the nucleotide sequences of the invention.

“LP229 variant polypeptide” or “LP229 variant” means an “active” LP229 polypeptide or fragment thereof as defined herein, having at least about 90% amino acid sequence identity with the LP229 polypeptides having the deduced amino acid sequences as shown in SEQ ID NO:2. Such LP229 polypeptide variants include, for instance, LP229 polypeptides wherein one or more amino acid residues are added, substituted or deleted, at the N- or C-terminus or within the sequence of SEQ ID NO:2. Ordinarily, an LP229 polypeptide variant will have at least about 90% amino acid sequence identity, preferably at least about 91% sequence identity, yet more preferably at least about 92% sequence identity, yet more preferably at least about 93% sequence identity, yet more preferably at least about 94% sequence identity, yet more preferably at least about 95% sequence identity, yet more preferably at least about 96% sequence identity, yet more preferably at least about 97% sequence identity, yet more preferably at least about 98% sequence identity, yet more preferably at least about 99% amino acid sequence identity with the amino acid sequence described, with or without the signal peptide.

Similarly, LP229 polynucleotides or polypeptides useful to practice the present invention may additionally contain other non-LP229 polynucleotide or polypeptide sequences, respectively, provided that the polypeptide encoded thereby still retains a functional activity. More specifically, LP229 polypeptides useful in practicing the present invention also include chimeric protein molecules not found in nature comprising a translational fusion, or in some cases an enzymatic fusion, in which two or more different proteins or fragments thereof are covalently linked on a single polypeptide chain. The fusion molecules are a subclass of chimeric polypeptide fusions of LP229 polypeptides that additionally contain a portion of an immunoglobulin sequence (herein referred to as “LP229-Ig”). The chimeric LP229-Ig fusions may also comprise forms in monomeric, homo- or heteromultimeric, and particularly homo- or heterodimeric, or homo- or heterotetrameric forms. Optionally, the chimeras may be in dimeric forms or homodimeric heavy chain forms. Tetrameric forms containing a four chain structural unit are the natural forms in which IgG, IgD, and IgE occur. A four-chain structure may also be repeated. Different chimeric forms containing a native immunoglobulin are known in the art (WO 98/25967). As used herein, the term “LP229-Ig” designates antibody-like molecules that combine at least one LP229 domain with the effector functions of immunoglobulin constant domain. The immunoglobulin constant domain sequence may be obtained from any immunoglobulin, such as IgG1, IgG2, IgG3 or IgG4 subtypes, IgA (including IgA1 and IgA2), IgE, IgD or IgM. LP229 fusion polypeptides can also comprise additional amino acid residues, such as affinity tags that aid in the purification or identification of the molecule or provide sites of attachment to a natural ligand.

The term “mature protein” or “mature polypeptide” as used herein refers to the form(s) of the protein produced by expression in a mammalian cell. It is generally hypothesized that once export of a growing protein chain across the rough endoplasmic reticulum has been initiated, proteins secreted by mammalian cells have a signal sequence that is cleaved from the complete polypeptide to produce a “mature” form of the protein. Oftentimes, cleavage of a secreted protein is not uniform and may result in more than one species of mature protein. The cleavage site of a secreted protein is determined by the primary amino acid sequence of the complete protein and generally cannot be predicted with complete accuracy. Methods for predicting whether a protein has a signal peptide sequence, as well as the cleavage point for that sequence, are available. A cleavage point may exist within the N-terminal domain between amino acid 10 and amino acid 35. More specifically the cleavage point is likely to exist after amino acid 15 but before amino acid 30, more likely after amino acid 24. As one of ordinary skill would appreciate, however, cleavage sites sometimes vary from organism to organism and cannot be predicted with absolute certainty. Optimally, cleavage sites for a secreted protein are determined experimentally by amino-terminal sequencing of the one or more species of mature proteins found within a purified preparation of the protein.

The term “modulate” means to affect (e.g., either upregulate, downregulate, or otherwise control) the level of a signaling pathway. Cellular processes under the control of signal transduction include, but are not limited to, transcription of specific genes, normal cellular functions, such as metabolism, proliferation, differentiation, adhesion, apoptosis and survival, as well as abnormal processes, such as transformation, blocking of differentiation and metastasis.

A “nucleic acid probe” or “probe” as used herein is a labeled nucleic acid compound that hybridizes with another nucleic acid compound. “Nucleic acid probe” means a single stranded nucleic acid sequence that will combine with a complementary or partially complementary single stranded target nucleic acid sequence to form a double-stranded molecule. A nucleic acid probe may be an oligonucleotide or a nucleotide polymer. A probe will usually contain a detectable moiety that may be attached to the end(s) of the probe or be internal to the sequence of the probe.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “patient” as used herein refers to any mammal, including humans, domestic and farm animals, zoo, sports or pet animals, such as cattle (e.g., cows), horses, dogs, sheep, pigs, rabbits, goats, cats, and non-domesticated animals like mice and rats. In a preferred embodiment of the invention, the mammal is a human or mouse.

“Percent (%) amino acid sequence identity” with respect to the LP229 amino acid sequences identified herein is defined as the percentage of amino acid residues in a candidate sequence that are identical with the amino acid residues in an LP229 polypeptide sequence, after aligning the sequences and introducing gaps, if necessary, to achieve the maximum percent sequence identity, and not considering any conservative amino acid substitutions as part of the sequence identity. Alignment for purposes of determining percent amino acid sequence identity can be achieved in various ways that are within the skill in the art, for instance, using publicly available computer software such as ALIGN, ALIGN-2, Megalign (DNASTAR) or BLAST (e.g., Blast, Blast-2, WU-Blast-2) software. Those skilled in the art can determine appropriate parameters for measuring alignment, including any algorithms needed to achieve maximal alignment over the full length of the sequences being compared. The percent identity values used herein are generated using WU-BLAST-2 (Altschul and Gish, Meth. Enzymol. 266: 460-80 (1996)). Most of the WU-BLAST-2 search parameters are set to the default values. Those not set to default values, i.e., the adjustable parameters, are set with the following values: overlap span=1; overlap fraction=0.125; word threshold (T)=11; and scoring matrix=BLOSUM 62. For purposes herein, a percent amino acid sequence identity value is determined by dividing (a) the number of matching identical amino acid residues between the amino acid sequence of the LP229 polypeptide of interest and the comparison amino acid sequence of interest (i.e., the sequence against which the LP229 polypeptide of interest is being compared) as determined by WU-BLAST-2, by (b) the total number of amino acid residues of the LP229 polypeptide of interest, respectively.

“Percent (%) nucleic acid sequence identity” with respect to the LP229 polynucleotide sequences identified herein is defined as the percentage of nucleotides in a candidate sequence that are identical with the nucleotides in the LP229 polynucleotide sequence after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as ALIGN, Align-2, Megalign (DNASTAR), or BLAST (e.g., Blast, Blast-2) software. Those skilled in the art can determine appropriate parameters for measuring alignment, including any algorithms needed to achieve maximal alignment over the full length of the sequences being compared. For purposes herein, however, percent nucleic acid identity values are generated using the WU-BLAST-2 (BlastN module) program (Altschul and Gish, Meth. Enzymol. 266: 460-80 (1996)). Most of the WU-BLAST-2 search parameters are set to the default values. Those not set default values, i.e., the adjustable parameters, are set with the following values: overlap span=1; overlap fraction=0.125; word threshold (T)=11; and scoring matrix=BLOSUM62. For purposes herein, a percent nucleic acid sequence identity value is determined by dividing (a) the number of matching identical nucleotides between the nucleic acid sequence of the LP229 polypeptide-encoding nucleic acid molecule of interest and the comparison nucleic acid molecule of interest (i.e., the sequence against which the LP229 polypeptide-encoding nucleic acid molecule of interest is being compared) as determined by WU-BLAST-2, by (b) the total number of nucleotides of the LP229 polypeptide-encoding nucleic acid molecule of interest.

“Pharmaceutically acceptable salt” includes, but is not limited to, salts prepared with inorganic acids, such as chloride, sulfate, phosphate, diphosphate, hydrobromide, and nitrate salts, or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

The term “plasmid” refers to an extrachromosomal genetic element. The plasmids disclosed herein are commercially available, publicly available on an unrestricted basis, or can be constructed from readily available plasmids in accordance with published procedures.

The term “positives,” in the context of sequence comparison performed as described above, includes residues in the sequences compared that are not identical but have similar properties (e.g., as a result of conservative substitutions). The percent identity value of positives is determined by the fraction of residues scoring a positive value in the BLOSUM 62 matrix. This value is determined by dividing (a) the number of amino acid residues scoring a positive value in the BLOSUM62 matrix of WU-BLAST-2 between the LP229 polypeptide amino acid sequence of interest and the comparison amino acid sequence (i.e., the amino acid sequence against which the LP229 polypeptide sequence is being compared) as determined by WU-BLAST-2, by (b) the total number of amino acid residues of the LP229 polypeptide of interest.

A “portion” of an LP229 polypeptide sequence is at least about 20, 25, 30, 35, 40, 45, 50, 55, 60, 65, 70, 75, 80, 85, 90, 95, or 100 contiguous amino acid residues in length.

A “primer” is a nucleic acid fragment which functions as an initiating substrate for enzymatic or synthetic elongation of, for example, a nucleic acid compound.

The term “promoter” refers to a nucleic acid sequence that directs transcription, for example, of DNA to RNA. An inducible promoter is one that is regulatable by environmental signals, such as carbon source, heat, or metal ions, for example. A constitutive promoter generally operates at a constant level and is not regulatable.

The term “recombinant DNA expression vector” or “expression vector” as used herein refers to any recombinant DNA cloning vector (such as a plasmid or phage), in which a promoter and other regulatory elements are present, thereby enabling transcription of an inserted DNA, which may encode a polypeptide.

“Single-chain Fvn” or “sFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domain, which enables the sFv to form the desired structure for antigen binding. For a review of sFv, see Pluckthun, The Pharmacology of Monoclonal Antibodies, vol. 113, Rosenburg and Moore, eds., Springer-Verlag, New York, pp. 269-315 (1994).

A “small molecule” is defined herein to have a molecular weight below about 500 daltons.

“Stringency” of hybridization reactions is readily determinable by one of ordinary skill in the art, and generally is an empirical calculation dependent upon probe length, washing temperature, and salt concentration. In general, longer nucleic acid probes required higher temperatures for proper annealing, while shorter nucleic acid probes need lower temperatures. Hybridization generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reactions more stringent, while lower temperatures less so. For additional details and explanation of stringency of hybridization reactions, see Ausubel, et al., Current Protocols in Molecular Biology, Wiley Interscience Publishers, 1995.

“Substantially pure,” when used in reference to an LP229 polynucleotide, polypeptide, or antibody means that said “LP229” is separated from other cellular and non-cellular molecules, including other proteins, lipids, carbohydrates or other materials with which it is naturally associated when produced recombinantly or synthesized without any general purifying steps. A “substantially pure” LP229 polypeptide described herein could be prepared by a variety of techniques well known to the skilled artisan, including, for example, the described methods of LP229 polypeptide purification referred to or described herein. In preferred embodiments, the LP229 polypeptide will be purified (1) to greater than 95% purity by weight of the LP229 polypeptide to the weight of total protein as determined by the Lowry method, and most preferably more than 99% by weight to the weight of total protein, (2) to apparent homogeneity by SDS-PAGE under reducing or nonreducing conditions using Coomassie Blue, or preferably, silver stain, such that the major band constitutes at least 95%, and more preferably 99%, of the stained protein observed on the gel.

The term “symptom” in reference to poor healing or chronic wounds, skin diseases, sepsis, inflammation, immunodeficiencies, autoimmune diseases, infectious diseases, allergic diseases, or malignancies is meant to include, but not limited to, one or more of the following: chills, profuse sweating, fever, weakness, hypotension, leukopenia, intravascular coagulation, shock, respiratory distress, organ failure, prostration, ruffled fur, diarrhea, eye exudate, and death, alone or in combination. This list is not meant to be exclusive, but may be supplemented with symptoms or combinations of symptoms that a person of ordinary skill would recognize as associated with poor healing or chronic wounds, skin diseases, sepsis, inflammation, immunodeficiencies, autoimmune diseases, infectious diseases, allergic diseases, or malignancies. Symptoms associated with these conditions that are treatable with LP229 are within the scope of this invention. A symptom associated with poor healing or chronic wounds, skin diseases, sepsis, inflammation, immunodeficiencies, autoimmune diseases, infectious diseases, allergic diseases, or malignancies may also be associated with another condition.

A “therapeutically-effective amount” is the minimal amount of active agent (e.g., an LP229 polypeptide, antagonist or agonist thereof) that is necessary to impart therapeutic benefit or desired biological effect to a patient. For example, a “therapeutically-effective amount” to a mammal suffering from sepsis is such an amount which induces, ameliorates or otherwise causes an improvement in the pathological symptoms, disease progression, physiological conditions associated with, or resistance to succumbing to a disorder principally characterized by poor healing or chronic wounds, skin diseases, sepsis, inflammation, immunodeficiencies, autoimmune diseases, infectious diseases, allergic diseases, and malignancies, particularly those disorders associated with the skin, when the LP229 polypeptide or LP229 epitope-recognizing antibody is administered. The precise amount of LP229 polypeptide or LP229 epitope-recognizing antibody administered to a particular patient will depend upon numerous factors, e.g., such as the specific binding activity of the molecule, the delivery device employed, physical characteristics, its intended use, and patient considerations, and can readily be determined by one skilled in the art, based upon the information provided herein and that which is known in the art.

The terms “treating,” “treatment,” and “therapy” as used herein refer to curative therapy, prophylactic therapy, and preventive therapy. An example of “preventive therapy” is the prevention or lessening of a targeted disease or related condition thereto. Those in need of treatment include those already with the disease or condition as well as those prone to have the disease or condition is to be prevented. The terms “treating”, “treatment”, and “therapy” as used herein also describe the management and care of a patient for the purpose of combating a disease or related condition, and includes the administration of LP229 to alleviate the symptoms or complications of said disease or condition.

The term “vector” as used herein refers to a nucleic acid compound used for introducing exogenous or endogenous DNA into host cells. A vector comprises a nucleotide sequence that may encode one or more protein molecules. Plasmids, cosmids, viruses, and bacteriophages, in the natural state or which have undergone recombinant engineering, are examples of commonly used vectors.

The various restriction enzymes disclosed and described herein are commercially available and the manner of use of said enzymes including reaction conditions, cofactors, and other requirements for activity are well known to one of ordinary skill in the art. Reaction conditions for particular enzymes are carried out according to the manufacturer's recommendation.

Protein Synthesis

Skilled artisans will recognize that the LP229 polypeptides utilized in the embodiments of the present invention can be synthesized by a number of different methods, such as chemical methods well known in the art, including solid phase peptide synthesis or recombinant methods. Both methods are described in U.S. Pat. No. 4,617,149, incorporated herein by reference.

The principles of solid phase chemical synthesis of polypeptides are well known in the art and may be found in general texts in the area. See, e.g., Dugas and Penney, Bioorganic Chemistry, Springer-Verlag, NY, 54-92 (1981). For example, peptides may be synthesized by solid-phase methodology utilizing an Applied Biosystems 430A peptide synthesizer (Applied Biosystems, Foster City, Calif.) and synthesis cycles supplied by Applied Biosystems.

The proteins utilized in the present invention can also be produced by recombinant DNA methods using the LP229 polynucleotide sequences provided herein. Recombinant methods are preferred if a high yield is desired. Expression of the LP229 polypeptide can be carried out in a variety of suitable host cells, well known to those skilled in the art. For this purpose, the LP229 polynucleotide constructs are introduced into a host cell by any suitable means, well known to those skilled in the art. Chromosomal integration of LP229 expression vectors are within the scope of the present invention, as well as suitable extra-chromosomally maintained expression vectors so that the coding region of the LP229 polynucleotide is operably-linked to a constitutive or inducible promoter.

The basic steps in the recombinant production of LP229 proteins are:

a) constructing a recombinant, synthetic or semi-synthetic DNA encoding LP229 protein;

b) integrating said DNA into an expression vector in a manner suitable for expressing the LP229 protein;

c) transforming or otherwise introducing said vector into an appropriate eukaryotic or prokaryotic host cell forming a recombinant host cell;

d) culturing said recombinant host cell in a manner to express the LP229 protein; and

e) recovering and substantially purifying the LP229 protein by any suitable means well known to those skilled in the art.

Production of LP229 products also include routes where direct chemical synthetic procedures are employed as well as products produced by recombinant techniques from a prokaryotic host or eukaryotic host, including, for example, yeast, higher plant, insect and mammalian cells. Depending upon the host employed in a recombinant production procedure, the polypeptides of the present invention can be glycosylated or can be non-glycosylated. Additionally, the amino acid sequence of an LP229 polypeptide may optionally include a conservative substitution. In addition, the LP229 polypeptides of the invention can also include an initial modified methionine residue, in some cases as a result of host-mediated processes. Such methods are described in many standard laboratory manuals, such as Sambrook, et al., Molecular Cloning: A Laboratory Manual, Cold Spring Laboratory Press, Cold Spring Harbor, N.Y. (1989), Chapters 17.37-17.42; Ausubel, supra, Chapters 10, 12, 13, 16, 18 and 20, entirely incorporated herein by reference.

Those skilled in the art will recognize that owing to the degeneracy of the genetic code (i.e., sixty-four codons that encode twenty amino acids), numerous “silent” substitutions of nucleotide base pairs could be introduced into an LP229 polynucleotide sequence without altering the identity of the encoded amino acid(s) or protein product. Use of all such substituted LP229 molecules is intended to be within the scope of the present invention.

Fragments of the proteins disclosed herein may be generated by any number of suitable techniques, including chemical synthesis. For instance constant regions of immunoglobulins can be obtained by papain digestion of antibodies. Alternatively, recombinant DNA mutagenesis techniques can provide LP229 molecules (see, e.g., Struhl, “Reverse biochemistry: Methods and applications for synthesizing yeast proteins in vitro,” Meth. Enzymol. 194: 520-35). For example, a nested set of deletion mutations are introduced into an LP229 polypeptide-encoding polynucleotide such that varying amounts of the protein coding region are deleted, either from the amino terminal end, or from the carboxyl end of the protein molecule. Further, additional changes or additions to the molecule can be made. This method can also be used to create internal fragments of the intact protein in which both the carboxyl and/or amino terminal ends are removed. Several appropriate nucleases can be used to create such deletions, for example Bal31, or in the case of a single stranded nucleic acid compound, mung bean nuclease. For simplicity, it is preferred that the intact LP229 gene be cloned into a single-stranded cloning vector, such as bacteriophage M13 or equivalent. If desired, the resulting gene deletion fragments can be subcloned into any suitable vector for propagation and expression of said fragments in any suitable host cell.

LP229 polypeptide can additionally be fused to a marker protein or an epitope tag. Such fusions include, but are not limited to, fusions to an enzyme, fluorescent protein, or luminescent protein that provides a marker function; or fusions to any amino acid sequence which can be employed for purification of the polypeptide or a proprotein sequence.

Methods of constructing fusion proteins (chimeras) composed of the binding domain of one protein and the constant region of an immunoglobulin (herein designated as “LP229-Ig”) are generally known in the art. For example, chimeras containing the Fc region of human IgG and the binding region of other protein receptors are known in the art for chimeric antibodies. LP229-Ig structures of the present invention can be constructed using methods similar to the construction of chimeric antibodies. In chimeric antibody construction, the variable domain of one antibody of one species is substituted for the variable domain of another species (see EP 0 125 023; EP 173 494; Munro, Nature 312(5995): 597 (1984); Neuberger, et al., Nature 312(5995): 604-8 (1984); Sharon, et al., Nature 309(5966): 364-7 (1984); Morrison and Oi, Annu. Rev. Immunol. 2: 239-56 (1984); Morrison, Science 229(4719): 1202-7 (1985); Boulianne, et al., Nature 312(5995): 643-6 (1984); Capon, et al., Nature 337(6207): 525-31 (1989); Traunecker, et al., Nature 339(6219): 68-70 (1989)). Here, a functional domain of the LP229 polypeptide is substituted for the variable domain of the recipient antibody structure.

Generally, methods for constructing LP229 fusion proteins include use of recombinant DNA technology. The DNA encoding a functional domain can optionally be fused with additional domains or segments of the LP229 polypeptide or with an Ig constant region. A polynucleotide encoding any domain of an LP229 polypeptide can be obtained by PCR or by restriction enzyme cleavage. This DNA fragment is readily inserted proximal to DNA encoding an immunoglobulin light or heavy chain constant region and, if necessary, the resulting construct is tailored by mutagenesis, to insert, delete, or change the codon sequence. Preferably, the selected immunoglobulin region is a human immunoglobulin region when the chimeric molecule is intended for in vivo therapy for humans. Most preferably, the selected immunoglobulin region is an IgG region. DNA encoding immunoglobulin light or heavy chain constant regions are known or readily available from cDNA libraries or can be synthesized. See, for example, Adams, et al., Biochemistry 19(12): 2711-9 (1980); Gough, et al., Biochemistry 19(12): 2702-10 (1980); Dolby, et al., Proc. Natl. Acad. Sci. USA 77(10): 6027-31 (1980); Rice and Baltimore, Proc. Natl. Acad. Sci. USA 79(24): 7862-5 (1982); Falkner and Zachau, et al., Nature 298(5871): 286-8 (1982); and Morrison and Oi, Annu. Rev. Immunol. 2: 239-56 (1984). Other teachings of preparing chimeric molecules are known from the preparation of immunoadhesin chimeras, such as CD4-Ig (Capon, et al., Nature 337(6207): 525-31 (1989); Byrn, et al., Nature, 344(6267): 667-70 (1990)) and TNFR chimeras, such as TNFR-IgG (Ashkenazi, et al., Proc. Natl. Acad. Sci. 88(23): 10535-9 (1991); Peppel, et al., J. Cell. Biochem. Supp. 15F-P439: 118 (1991)).

Protein Purification

Generally, LP229 polypeptides are produced recombinantly. Once expressed, they can be isolated from the cells by applying standard protein isolation techniques to the lysates or purified from the media. The monitoring of the purification process can be accomplished by using standard Western blot techniques or radioimmunoassays or other standard immunoassay techniques.

LP229 polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, size exclusion chromatography, and lectin chromatography. Preferably, high performance liquid chromatography (“HPLC”), cation exchange chromatography, affinity chromatography, size exclusion chromatography, or combinations thereof, are employed for purification. Particular methods of using protein A or G chromatography for purification are known in the art and are particularly applicable where the LP229 polypeptide contains an immunoglobulin Fc region. Protein A and G binds the Fc regions of IgG antibodies and, therefore, makes a convenient tool for the purification of LP229 polypeptides containing the IgG region. LP229 polypeptide purification is meant to include purified parts of the chimera that are purified separately and then combined by disulfide bonding, cross-linking or the like.

The purification of LP229 polypeptides can be accomplished by a number of special techniques known in the art that take particular advantage of structural and functional features of these molecules. See, e.g., Kwon, et al., J. Biol. Chem. 272 (22): 14272-6 (1997); Emery, et al., J. Biol. Chem. 273(42): 14363-7 (1998); Harrop, et al., J. Biol. Chem. 273(42): 27548-56 (1998); Harrop, et al., J. Immunol. 161(4): 1786-94 (1998). Further, a number of advantageous protein sequences can be incorporated into the LP229 polypeptide produced, such as factor Xa cleavage sites, a HIS tag sequence, or the incorporation of specific epitopes, as is known in the art.

Expressing Recombinant LP229 Proteins in Host Cells

Prokaryotes may be employed in the production of recombinant LP229 proteins. For example, the Escherichia coli K12 strain 294 (ATCC 31446) is particularly useful for the prokaryotic expression of foreign proteins. Other strains of E. coli, bacilli such as Bacillus subtilis, and other bacteria, such as Streptomyces, may also be employed as host cells in the cloning and expression of the recombinant proteins of this invention.

Promoter sequences suitable for driving the expression of genes in prokaryotes include beta-lactamase (e.g., vector pGX2907, ATCC 39344, contains a replicon and beta-lactamase gene), lactose systems (Chang, et al., Nature (London) 275: 615 (1978); Goeddel, et al., Nature (London) 281: 544 (1979)), alkaline phosphatase, and the tryptophan (trp) promoter system (vector pATH1, ATCC 37695), which is designed to facilitate expression of an open reading frame as a trpE fusion protein under the control of the trp promoter. Hybrid promoters such as the tac promoter (isolatable from plasmid pDR540, ATCC 37282) are also suitable. Still other bacterial promoters, whose nucleotide sequences are generally known, may be ligated to DNA encoding the protein of the instant invention, using linkers or adapters to supply any required restriction sites. Promoters for use in bacterial systems also will contain a Shine-Dalgarno sequence operably linked to the DNA encoding the desired polypeptides. These examples are illustrative rather than limiting.

The LP229 proteins required to practice the present invention may be synthesized either by direct expression or as a fusion protein comprising the protein of interest as a translational fusion with another protein or peptide that may be removed by enzymatic or chemical cleavage. It is often observed in the production of certain peptides in recombinant systems that expression as containing other desired sequences prolongs the lifespan, increases the yield of the desired peptide, or provides a convenient means of isolating the protein. This is particularly relevant when expressing mammalian proteins in prokaryotic hosts. A variety of peptidases (e.g., enterokinase and thrombin) that cleave a polypeptide at specific sites or digest the peptides from the amino or carboxy termini (e.g., diaminopeptidase) of the peptide chain are known. Furthermore, particular chemicals (e.g., cyanogen bromide) will cleave a polypeptide chain at specific sites. The skilled artisan will appreciate the modifications necessary to the amino acid sequence (and synthetic or semi-synthetic coding sequence if recombinant means are employed) to incorporate site-specific internal cleavage sites. See e.g., Carter, “Site Specific Proteolysis of Fusion Proteins”, Chapter 13, in Protein Purification: From Molecular Mechanisms to Large Scale Processes, American Chemical Society, Washington, D.C. (1990).

In addition to prokaryotes mammalian cell systems can be used. The choice of a particular host cell depends to some extent on the particular expression vector used. Exemplary mammalian host cells suitable for use in the present invention include 293 (e.g., ATCC CCL 1573), 3T3 (ATCC CCL 92), CHO-K1 (ATCC CCL 61), COS and BHK-21 (ATCC CCL 10), for example.

A wide variety of vectors are suitable for transforming mammalian host cells. For example, the pSV2-type vectors comprise segments of the simian virus 40 (SV40) genome required for transcription and polyadenylation. A large number of plasmid pSV2-type vectors have been constructed, such as pSV2-gpt, pSV2-neo, pSV2-dhfr, pSV2-hyg, and pSV2-beta-globin, in which the SV40 promoter drives transcription of an inserted gene. These vectors are widely available from sources such as the American Type Culture Collection (ATCC), Rockville, Md., or the National Center for Agricultural Utilization Research, Peoria, Illinois.

Promoters suitable for expression in mammalian cells include the SV40 late promoter, promoters from eukaryotic genes, such as, for example, the estrogen-inducible chicken ovalbumin gene, the interferon genes, the glucocorticoid-inducible tyrosine aminotransferase gene, the thymidine kinase gene promoter, and the promoters of the major early and late adenovirus genes.

Plasmid pRSVcat (ATCC 37152) comprises portions of a long terminal repeat of the Rous sarcoma virus, a virus known to infect chickens and other host cells. This long terminal repeat contains a promoter that is suitable for this use. (Gorman, et al., Proc. Nat. Acad. Sci. USA 79(22): 6777-81 (1982)). The plasmid pMSVi (NRRL B-15929) comprises the long terminal repeats of the Murine Sarcoma virus, a virus known to infect mouse and other host cells. The mouse metallothionein promoter has also been well characterized for use in eukaryotic host cells and is suitable for use in the present invention. This promoter is present in the plasmid pdBPV-MMTneo (ATCC 37224) that can serve as the starting material for the construction of other expression plasmids that would also be useful in producing LP229 polypeptides.

Transfection of mammalian cells with vectors can be performed by a plurality of well-known processes including, but not limited to, protoplast fusion, calcium phosphate co-precipitation, electroporation and the like. See, e.g., Maniatis, et al., supra.

Some viruses also make appropriate vectors. Examples include the adenoviruses, the adeno-associated viruses, the vaccinia virus, the herpes viruses, the baculoviruses, and the Rous Sarcoma virus, as described in U.S. Pat. No. 4,775,624, incorporated herein by reference.

Eukaryotic microorganisms such as yeast and other fungi are also suitable host cells. The yeast Saccharomyces cerevisiae is the preferred eukaryotic microorganism. Other yeasts such as Kluyveromyces lactis and Pichia pastoris are also suitable. For expression in Saccharomyces, the plasmid YRp7 (ATCC 40053), for example, may be used. See, e.g., Stinchcomb, et al., Nature 282(5734): 39-43 (1979); Kingsman, et al., Gene 7(2): 141-52 (1979); Tschumper and Carbon, Gene 10(2): 157-66 (1980). Plasmid YRp7 contains the TRP1 gene that provides a selectable marker for use in a trpl auxotrophic mutant.

Production of Antibodies

The methods of the present invention may also rely on use of LP229 epitope-recognizing antibodies to treat various conditions relating to skin diseases, sepsis, inflammation, immunodeficiencies, autoimmune diseases, infectious diseases, allergic diseases, and malignancies, particularly those disorders associated with the skin. The production of antibodies, including both monoclonal and polyclonal, in animals, especially mice, is well known in the art. See, e.g., Milstein, Handbook of Experimental Immunology, Blackwell Scientific Pub. (1986); Goding, Monoclonal Antibodies: Principles and Practice, Academic Press (1983). For the production of monoclonal antibodies the basic process begins with injecting a mouse, or other suitable animal, with an immunogen. The mouse is subsequently sacrificed and cells taken from its spleen are fused with myeloma cells, resulting in a hybridoma that reproduces in vitro. The population of hybridomas is screened to isolate individual clones, each of which secretes a single antibody species, specific for the immunogen. Each antibody obtained in this way is the clonal product of a single B cell.

Chimeric antibodies are described in U.S. Pat. No. 4,816,567, the entire contents of which are herein incorporated by reference. This reference discloses methods and vectors for the preparation of chimeric antibodies. An alternative approach is provided in U.S. Pat. No. 4,816,397, the entire contents of which are herein incorporated by reference. This patent teaches co-expression of the heavy and light chains of an antibody in the same host cell.

The approach of U.S. Pat. No. 4,816,397 has been further refined in European Patent Publication 0 239 400. The teachings of this European patent publication are a preferred format for genetic engineering of monoclonal antibodies. In this technology the complementarily determining regions (CDRs) of a human antibody are replaced with the CDRs of a murine monoclonal antibody, thereby converting the specificity of the human antibody to the specificity of a murine antibody.

Single chain antibodies and libraries thereof are yet another variety of genetically engineered antibody technology that is well known in the art. (See, e.g., Bird, et al., Science 242(4877): 423-6 (1988); WO 88/01649, WO 90/14430, and WO 91/10737). Single chain antibody technology involves covalently joining the binding regions of heavy and light chains to generate a single polypeptide chain. The binding specificity of the intact antibody molecule is thereby reproduced on a single polypeptide chain.

The proteins or suitable fragments thereof required to generate polyclonal or monoclonal antibodies, and various interspecies hybrids, or humanized antibodies, or antibody fragments, or single-chain antibodies are disclosed herein. The techniques for producing antibodies are well known to skilled artisans. See, e.g., Campbell, Monoclonal Antibody Technology: Laboratory Techniques in Biochemistry and Molecular Biology, Elsevier Science Publishers, Amsterdam (1984); Kohler and Milstein, Nature 256(5517): 495-7 (1975); Monoclonal Antibodies: Principles & Applications, Eds. Birch and Lennox, Wiley-Liss (1995).

The most preferred method of generating MAbs to the polypeptides and glycopeptides of the present invention comprises producing said MAbs in a transgenic mammal modified in such a way that they are capable of producing fully human MAbs upon antigenic challenge. Human MAbs and methods for their production are generally known in the art (see, e.g., U.S. Pat. Nos. 4,704,362, 4,816,567, 5,434,340, 5,545,806, 5,530,101, 5,569,825, 5,585,089, 5,625,126, 5,633,425, 5,643,763, 5,693,761, 5,693,762, 5,714,350, 5,874,299, 5,877,397, 5,939,598, 6,023,010, 6,054,297, WO 96/34096, WO 96/33735, and WO 98/24893).

A protein used as an immunogen may be modified or administered in an adjuvant, by subcutaneous or intraperitoneal injection into, for example, a mouse or a rabbit. For the production of monoclonal antibodies, spleen cells from immunized animals are removed, fused with myeloma cells, such as SP2/0-Ag14 cells, and allowed to become monoclonal antibody producing hybridoma cells in the manner known to the skilled artisan. Hybridomas that secrete a desired antibody molecule can be screened by a variety of well known methods, for example ELISA assay, western blot analysis, or radioimmunoassay (Lutz, et al., Exp. Cell Res. 175(1): 109-24 (1988); Monoclonal Antibodies: Principles & Applications, Eds. Birch and Lennox, Wiley-Liss (1995)).

Nucleic Acids

The synthesis of the LP229 polynucleotides (such as provided in SEQ ID NO:1) and related nucleic acids that would encode LP229 polypeptides as defined herein or fragments thereof is well known in the art. See, e.g., Brown, et al., Meth. Enzymol. 68: 109-51 (1979). Fragments of the DNA sequence corresponding to LP229 sequences could be generated using a conventional DNA synthesizing apparatus, such as the Applied Biosystems Model 380A or 380B DNA synthesizers (Applied Biosystems, Inc., Foster City, Calif.) using phosphoramidite chemistry, thereafter ligating the fragments so as to reconstitute the entire LP229 sequence. Alternatively, phosphotriester chemistry may be employed to synthesize the nucleic acids of this invention. See, e.g., Gait, ed., Oligonucleotide Synthesis, A Practical Approach (1984).

In an alternative methodology, namely PCR, the DNA sequences disclosed and described herein, comprising, for example, a portion or all of SEQ ID NO:1, can be produced from a plurality of starting materials. For example, starting with a cDNA preparation (e.g., cDNA library) derived from a tissue that expresses the LP229 gene, suitable oligonucleotide primers complementary to regions of SEQ ID NO:1 or to any sub-region therein, are prepared as described in U.S. Pat. No. 4,889,818, hereby incorporated by reference. Other suitable protocols for the PCR are disclosed in PCR Protocols: A Guide to Method and Applications, Innis, et al., Academic Press, Inc. (1990). Using PCR, any region of the LP229 gene can be targeted for amplification such that full or partial length gene sequences containing a functional domain may be produced.

In certain embodiments, it is advantageous to use oligonucleotide primers. The sequence of such primers is designed using a polynucleotide of the present invention for use in detecting, amplifying, or mutating a defined segment of a gene or polynucleotide that encodes an LP229 polypeptide using PCR technology.

The present disclosure provides exemplary methods for constructing a recombinant host cell capable of expressing proteins comprising LP229 polypeptides, said method comprising transforming or otherwise introducing into a host cell a recombinant DNA vector that comprises an isolated DNA sequence that encodes polypeptides comprising sequences as shown in SEQ ID NO:2 or fragments thereof. The preferred host cell is any eukaryotic cell that can accommodate high-level expression of an exogenously introduced gene or protein. The skilled artisan understands that choosing the most appropriate cloning vector or expression vector depends upon a number of factors including the availability of restriction enzyme sites, the type of host cell into which the vector is to be transfected or transformed, the purpose of the transfection or transformation (e.g., stable transformation as an extrachromosomal element, or integration into the host chromosome), the presence or absence of readily assayable or selectable markers (e.g., antibiotic resistance and metabolic markers of one type and another), and the number of copies of the gene desired in the host cell.

When preparing an expression vector the skilled artisan understands that there are many variables to be considered, for example, whether to use a constitutive or inducible promoter. The practitioner also understands that the amount of nucleic acid or protein to be produced dictates, in part, the selection of the expression system. Regarding promoter sequences, inducible promoters are preferred because they enable high level, regulatable expression of an operably linked gene. The skilled artisan will recognize a number of suitable promoters that respond to a variety of inducers, for example, carbon source, metal ions, and heat. Other relevant considerations regarding an expression vector include whether to include sequences for directing the localization of a recombinant protein. For example, a sequence encoding a signal peptide preceding the coding region of a gene is useful for directing the extracellular export of a resulting polypeptide. Transformed host cells may be cultured under conditions well known to skilled artisans such that a polypeptide comprising sequence as shown in SEQ ID NO:2 is expressed, thereby producing a recombinant LP229 protein in the recombinant host cell.

Transgenic and Chimeric Non-Human Mammals

Nucleic acids which encode an LP229 polypeptide of the present invention or any of its modified forms can also be used to generate either transgenic animals or “knock out” animals which, in turn, are useful in the development and screening of therapeutically useful reagents. Methods for generating transgenic animals, particularly animals such as mice or rats, have become conventional in the art and are described, for example, in U.S. Pat. No. 4,736,866 and 4,870,009. Typically, particular cells would be targeted for an LP229 transgene incorporation with tissue-specific enhancers. Transgenic animals that include a copy of a transgene introduced into the germ line of the animal at an embryonic stage can be used to examine the effect of increased expression of DNA encoding an LP229 polypeptide. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologs of LP229 polynucleotides can be used to construct a “knock out” animal which has a defective or altered gene encoding a particular LP229 polypeptide as a result of homologous recombination between the endogenous gene encoding the LP229 polypeptide and the altered genomic DNA introduced into an embryonic cell of the animal. For example, cDNA encoding an LP229 polypeptide can be used to clone genomic DNA encoding that LP229 polypeptide in accordance with established techniques. A portion of the genomic DNA encoding an LP229 polypeptide can be deleted or replaced with another gene, such as a gene encoding a selectable marker that can be used to monitor integration. Typically, several kilobases of unaltered flanking DNA (both at the 5′ and 3′ ends) are included in the vector (see, e.g., Thomas and Capecchi, Cell 51(3): 503-12 (1987) for a description of homologous recombination vectors). The vector is introduced into an embryonic stem cell line (e.g., by electroporation), and cells in which the introduced DNA has homologously recombined with the endogenous DNA are selected (see, e.g., Li, et al., Cell 69(6): 915-26 (1992)). The selected cells are then injected into a blastocyst of an animal (e.g., a mouse or rat) to form aggregation chimeras (see, e.g., Bradley, Teratocarcinomas and Embryonic Stem Cells: A Practical Approach, Robertson, ed. (IRL, Oxford, 1987), pp. 113-52). A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized, for instance, for their ability to defend against certain pathological conditions and for their development of pathological conditions due to absence of the native LP229 polypeptide.

Transgenic non-human mammals are useful as animal models in both basic research and drug development endeavors. Transgenic animals expressing at least one LP229 polypeptide or nucleic acid can be used to test compounds or other treatment modalities that may prevent, suppress, or cure a pathology or disease associated with at least one of the above-mentioned activities. Such transgenic animals can also serve as a model for the testing of diagnostic methods for those same diseases. Furthermore, tissues derived from such transgenic non-human mammals are useful as a source of cells for cell culture in efforts to develop in vitro bioassays to identify compounds that modulate LP229 polypeptide activity or LP229 polypeptide dependent signaling. Accordingly, another aspect of the present invention contemplates a method of identifying compounds efficacious in the treatment of at least one previously described disease or pathology associated with LP229 polypeptide associated activity. A non-limiting example of such a method comprises:

a) generating a transgenic non-human animal that expresses an LP229 polypeptide of the present invention and which is, as compared to a wild-type animal, pathologically distinct in some detectable or measurable manner from wild-type version of said non-human mammal;

b) exposing said transgenic animal to a compound, and;

c) determining the progression of the pathology in the treated transgenic animal, wherein an arrest, delay, or reversal in disease progression in transgenic animal treated with said compound as compared to the progression of the pathology in an untreated control animal is indicative that the compound is useful for the treatment of said pathology.

Another embodiment of the present invention provides a method of identifying compounds capable of inhibiting LP229 polypeptide activity in vivo and/or in vitro wherein said method comprises:

a) administering an experimental compound to an LP229 polypeptide-expressing transgenic non-human animal or tissues derived therefrom, exhibiting one or more physiological or pathological conditions attributable to the expression of an LP229 transgene; and

b) observing or assaying said animal and/or animal tissues to detect changes in said physiological or pathological condition or conditions.

Another embodiment of the invention provides a method for identifying compounds capable of overcoming deficiencies in LP229 polypeptide activity in vivo or in vitro wherein said method comprises:

a) administering an experimental compound to an LP229 polypeptide-expressing transgenic non-human animal, or tissues derived therefrom, exhibiting one or more physiological or pathological conditions attributable to the disruption of the endogenous LP229 polypeptide-encoding gene; and

b) observing or assaying said animal and/or animal tissues to detect changes in said physiological or pathological condition or conditions.

Various means for determining a compound's ability to modulate the activity of an LP229 polypeptide in the body of the transgenic animal are consistent with the invention. Observing the reversal of a pathological condition in the LP229 polypeptide expressing transgenic animal after administering a compound is one such means. Another more preferred means is to assay for markers of LP229 activity in the blood of a transgenic animal before and after administering an experimental compound to the animal. The level of skill of an artisan in the relevant arts readily provides the practitioner with numerous methods for assaying physiological changes related to therapeutic modulation of LP229 activity.

“Gene therapy” includes both conventional gene therapies, where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has been shown that short antisense oligonucleotides can be imported into cells where they act as inhibitors, despite their low intracellular concentrations caused by their restricted uptake by the cell membrane (Zamecnik, et al., Proc. Natl. Acad Sci. USA 83(12): 4143-6 (1986)). The oligonucleotides can be modified to enhance their uptake, e.g., by substituting their negatively charged phosphodiester groups with uncharged groups.

There are a variety of techniques available for introducing nucleic acids into viable cells. The techniques vary depending upon whether the nucleic acid is transferred into cultured cell in vitro or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically, retroviral) vectors and viral coat protein-liposome mediated transfection (Dzau, et al., Trends in Biotechnology 11(5): 205-10 (1993)). In some situations it is desirable to provide the nucleic acid source with an agent that targets the target cells, such as an antibody specific for a cell surface membrane protein or the target cell, a ligand for a receptor on the target cells, etc. Where liposomes are employed, proteins which bind to a cell surface membrane protein associated with endocytosis may by used for targeting and/or to facilitate uptake, e.g., capsid proteins or fragments thereof trophic for a particular cell type, antibodies for proteins which undergo internalization in cycling, proteins that target intracellular localization and enhance intracellular half-life. The technique of receptor-mediated endocytosis is described, for example by Wu, et al., J. Biol. Chem. 262(10): 4429-32 (1987); and Wagner, et al., Proc. Natl. Acad. Sci. USA 87(9): 3410-4 (1990). For a review of gene marking and gene therapy protocols, see Anderson, Science 256(5058): 808-13 (1992).

Methods of Treatment Using LP229 Polypeptides

Data presented in Examples 3 and 4 show that sepsis, inflammation, and conditions or symptoms related thereto may be treated or prevented by administration of effective amounts of LP229 polypeptides. Administration of LP229 inhibited the effects occurring during acute endotoxic shock and prevented death. As characterized generally, the invention also relates to methods preventing or treating conditions caused or exacerbated by poor healing or chronic wounds, skin diseases, sepsis, inflammation, immunodeficiencies, autoimmune diseases, infectious diseases, allergic diseases, and malignancies, particularly those disorders associated with the skin, and conditions or symptoms related thereto by administering LP229 polypeptides, variants or active fragments thereof. Additionally, the invention relates to methods preventing or treating conditions including, but not limited to, skin diseases, sepsis, inflammation, immunodeficiencies, autoimmune diseases, infectious diseases, allergic diseases, and malignancies, particularly those disorders associated with the skin, by administering LP229 epitope-recognizing antibodies.

Substantially pure or purified preparations of LP229 polypeptides or LP229 epitope-recognizing antibodies can be formulated into a pharmaceutically acceptable composition. Such formulations can be dosed in a fashion consistent with good medical practice, taking into account the clinical condition of the individual patient (especially the side effects of treatment with LP229 polypeptides or LP229 epitope-recognizing antibodies alone), the site of delivery of the LP229 polypeptides or LP229 epitope-recognizing antibody compositions, the method of administration, the scheduling of administration, and other factors known to practitioners.

An effective amount of an LP229 polypeptide or LP229 epitope-recognizing antibody will serve to prevent or treat at least one symptom of poor healing or chronic wounds, skin diseases, sepsis, inflammation, immunodeficiencies, autoimmune diseases, infectious diseases, allergic diseases, and malignancies, particularly those disorders associated with the skin, or will serve to modulate the biological activity. An effective amount of an LP229 polypeptide or an LP229 epitope-recognizing antibody to prevent or treat at least one symptom may be determined by prevention or amelioration of adverse conditions or symptoms of the diseases being treated. The therapeutically effective amount of an LP229 polypeptide or an LP229 epitope-recognizing antibody for purposes herein is thus determined by such considerations. By delivery of graduating levels of LP229 polypeptide or LP229 epitope-recognizing antibody within a pharmaceutical composition, a clinician skilled in the art can determine the therapeutically effective dose of an LP229 polypeptide or LP229 epitope-recognizing antibody for treatment or prevention of poor healing or chronic wounds, skin diseases, sepsis, inflammation, immunodeficiencies, autoimmune diseases, infectious diseases, allergic diseases, and malignancies, particularly those disorders associated with the skin. Such determinations are well known in the art and within the skill of the clinician in adjusting the therapeutically effective amount of LP229 polypeptide or LP229 epitope-recognizing antibody in a pharmaceutical composition accordingly. A therapeutically effective amount of LP229 polypeptide or LP229 epitope-recognizing antibody results in a measurable modulation of the biological activity associated with LP229 polypeptide.

As a general proposition, the total therapeutically effective amount of LP229 polypeptide or LP229 epitope-recognizing antibody administered parentally per dose of a pharmaceutical composition will be in the range of about 1 μg/kg/day to 10 mg/kg/day of patient body weight. However, as noted above, this will be subject to therapeutic discretion. Preferably, this dose is at least 0.001 mg/kg/day, or at least 0.01 mg/kg/day, or at least 0.10 mg/kg/day, or at least 1.0 mg/kg/day.

As a further proposition, if given continuously, the LP229 polypeptide or LP229 epitope-recognizing antibody is typically administered at a dose rate of about 0.1 μg/kg/hour to about 50 μg/kg/hour, either by one to four injections per day or by continuous subcutaneous infusions, for example, using a mini-pump. An intravenous bag solution may also be employed. The length of treatment needed to observe changes and the interval following treatment for responses to occur appear to vary depending on the desired effect.

Pharmaceutical compositions containing an LP229 polypeptide or LP229 epitope-recognizing antibody may be administered using a variety of modes that include, but are not limited to, oral, rectal, intra-cranial, parenteral, intracisternal, intrathecal, intravaginal, intraperitoneal, intratracheal, intrabroncho-pulmonary, topical, transdermal (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers to modes of administration that include, but are not limited to, intravenous, intramuscular, intraperitoneal, intrasternal, subcutaneous and intraarticular injection and infusion. Implants comprising an LP229 polypeptide or an LP229 epitope-recognizing antibody also can be used.

LP229 polypeptides or LP229 epitope-recognizing antibodies are also suitably administered by sustained-release systems. Suitable examples of sustained-release compositions include semi-permeable polymer matrices, e.g., films, or microcapsules. Sustained-release matrices include polylactides (U.S. Pat. No. 3,773,919, EP 058 481), copolymers of L-glutamic acid and gamma-ethyl-L-glutamate (Sidman, et al., Biopolymers 22: 547-56 (1983)), poly-(2-hydroxyethyl-methacrylate) (Langer, et al., J. Biomed. Matl. Res. 15: 167-277 (1981)), ethylene vinyl acetate (Langer, et al., 1982) or poly-D-3-hydroxybutyric acid (EP 133 988).

Sustained-release LP229 polypeptide or LP229 epitope-recognizing antibody compositions also include liposomally entrapped LP229 polypeptides. Liposomes containing LP229 polypeptides are prepared by methods known per se (DE 3 218 121; Epstein, et al., Proc. Natl. Acad. Sci. USA 82: 3688-92 (1985); Hwang, et al., Proc. Natl. Acad. Sci. USA 77: 4030-4 (1980); EP 52 322; EP 36 676; EP 88 046; EP 143 949; EP 142 641; Japanese Patent Application 83-118008; U.S. Pat. Nos. 4,485,045 and 4,544,545; and EP 102 324. Ordinarily, the liposomes are of the small (about 200 to 800 Angstroms) unilamellar type in which the lipid content is greater than about 30 mol percent cholesterol, the selected proportion being adjusted for the optimal LP229 polypeptide therapy.

For parenteral administration, in one embodiment, the LP229 polypeptide or LP229 epitope-recognizing antibody is formulated generally by mixing at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting the LP229 polypeptide uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG.

The LP229 polypeptide or LP229 epitope-recognizing antibody is typically formulated in such vehicles at a concentration of about 0.1 mg/mL to 100 mg/mL, preferably 1 to 10 mg/mL, at a pH of about three to eight. It will be understood that the use of certain of the foregoing excipients, carriers, or stabilizers will result in the formation of LP229 polypeptide salts. Pharmaceutical compositions comprising LP229 polypeptides or LP229 epitope-recognizing antibodies to be used for therapeutic administration must be sterile. Sterility is readily accomplished by filtration through sterile filtration membranes (e.g., 0.2 micron membranes). Pharmaceutical compositions comprising LP229 polypeptides or LP229 epitope-recognizing antibodies generally are placed into a container having a sterile access port, for example, an intravenous solution bag or vial having a stopper pierceable by a hypodermic injection needle.

Pharmaceutical compositions comprising LP229 polypeptides or LP229 epitope-recognizing antibodies ordinarily will be stored in unit or multi-dose containers, for example, sealed ampoules or vials, as an aqueous solution or as a lyophilized formulation for reconstitution. As an example of a lyophilized formulation, ten-milliliter vials are filled with five milliliters sterile-filtered 1% (w/v) aqueous LP229 polypeptide solution, and the resulting mixture is lyophilized. The infusion solution is prepared by reconstituting the lyophilized LP229 polypeptide using bacteriostatic water-for-injection.

In addition, the present invention includes methods for the treatment or prevention of poor healing or chronic wounds, skin diseases, sepsis, inflammation, immunodeficiencies, autoimmune diseases, infectious diseases, allergic diseases, and malignancies, particularly those disorders associated with the skin, and conditions or symptoms related thereto, comprising administering pharmaceutical compositions comprising LP229 polypeptides or LP229 epitope-recognizing antibodies to a patient in need of such therapy wherein said composition further comprises other therapeutic compounds.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present application, including definitions, will control. In addition, the materials, methods and examples described herein are illustrative only and not intended to be limiting.

The following examples more fully describe the present invention.

### EXAMPLES

### Example 1

### Generation and Affinity Purification of Anti-LP229 Antisera

Polyclonal antiserum can be prepared by well-known methods (e.g., Vaitukaitis, et al., J. Clin. Endocrinol. Metab. 33(6): 988-91 (1971)) that involve immunizing suitable animals with one or more of the LP229 polypeptides, or fragments thereof, disclosed herein. Rabbits are immunized with 50 μg LP229 emulsified with Complete Freund's adjuvant for the primary immunization and antigen plus Incomplete Freund's adjuvant for two subsequent immunizations. The resulting antisera are Protein A purified followed by affinity purification against LP229 coupled to resin.

### Example 2

### Immunohistochemistry Using Anti-LP229 Antisera

Immunohistochemistry is performed using a Dako automated immunostaining machine. Briefly, the slides are deparaffinized, followed by treatment for twenty minutes with Dako antigen retrieval solution. The slides are blocked for forty-five minutes, followed by addition of anti-LP229 at 10 μg/mL for two hours. The slides are washed, exposed to biotinylated secondary antibody, and developed using a DAB substrate. The slides are then counterstained with hematoxylin.

LP229 is specifically expressed in the epithelium, sweat glands, and hair follicles of the skin. It is also expressed in the Kupfer cells in the liver, in macrophages/dendritic cells in the spleen, and in the Paneth cells in the duodenum.

### Example 3

### LP229 Protects Against LPS-Induced Septic Shock in Mice

This example demonstrates that injecting LP229 DNA can protect against LPS-induced septic shock in mice. Eight to ten-week old BALB/c mice (Harlan, Indianapolis) are given 20 μg/mouse of human LP229 DNA by intravenous injection (lateral tail vein). Twenty-four hours later, 175 μg LPS are injected intravenously into each mouse to induce sepsis. Control mice are similarly injected with the exception that vector DNA, as opposed to LP229 DNA, is injected into one control group and IL-10 DNA is injected into another group.

The mice are monitored for 120 hours to determine survival. LP229 injected mice have a 100% survival rate after 120 hours, compared to vector injected mice which have a 0% survival rate after only twenty-four hours. IL-10 injected mice also have a 100% survival rate.

### Example 4

### LP229 Inhibits LPS-Induced Increases in IL-1-beta, TNF-alpha, and IFN-gamma Secretion

During endotoxemia, cytokines are central players in the disease process (Dinarello, Curr. Top. Microbiol. Immunol. 216: 133-65 (1996)). The pro-inflammatory cytokines IL-12, IFN-gamma, and TNF-alpha are key cytokines of the generalized Shwartzman reaction (Ozmen, et al., J. Exp. Med. 180(3): 907-15 (1994)). TNF-alpha, IL-12 and IFN-gamma are rapidly induced following LPS administration, and these factors all contribute to endotoxin-induced lethality. IFN-gamma appears to act by promoting increased responsiveness to TNF, in part by enhancing synthesis of both TNF and its receptors (Doherty, et al., J. Immunol. 149(5): 1666-70 (1992)). IL-12 also can play a critical role during endotoxin shock, primarily through its function as an inducer of IFN-gamma (Wysocka, et al., Eur. J. Immunol. 25(3): 672-6 (1995)).

In this experiment, 20 μg of LP229 DNA is injected into six male BALB/c mice. Approximately twenty-four hours later, each mouse is challenged with 175 μg E. coli LPS by intravenous injection. This dose of LPS causes acute inflammation in mice and 90 to 100% death rate, based on prior experiments. In response to LPS treatment, many pro-inflammatory cytokines are synthesized during the first several hours. At two hours post LPS injection, three mice are bled retro-orbitally, collecting 200 μL serum for cytokine analysis. This procedure is repeated six hours post LPS injection using the other three mice. Approximately twenty-four hours post injection, all mice are terminally bled via cardiac puncture after carbon dioxide asphyxiation, and 200 μL of serum are collected for clinical chemistry analysis. Control mice follow the same procedure, injecting vector DNA as opposed to LP229 in the initial injection. Human IL-10 DNA is injected into a third group of mice as a positive control.

Serum samples taken at the two- and six-hour post LPS injection time points are analyzed by ELISA. Vector DNA injections yield elevated serum levels of TNF-alpha and IL-10 at two hours post LPS injection and elevated serum levels of IL-1-beta, IL-10, and IFN-gamma at six hours post LPS injection. Human IL-10 DNA injected controls yield significantly lower serum levels of TNF-alpha, IL-1-beta, and IL-10.

LP229 DNA-injected mice have lower levels of IL-1-beta, TNF-alpha, and IFN-gamma. LP229 lowers IL-1-beta and TNF-alpha slightly less effectively than the IL-10 control. LP229 lowers IFN-gamma more effectively than the IL-10 control. LP229-injected mice have increased levels of GM-CSF but yield no change in the level of IL-10. The evidence provided here demonstrates that administration of LP229 polypeptides can inhibit inflammation and may be an effective therapy in treating human inflammatory diseases.

### Example 5

### LP229 Exhibits Antibacterial Activity by Inhibiting Growth of E. coli and S. aureus

SLP1 and LP229 are tested for antibacterial activity in a “Kill Curve Assay.” Two bacterial strains, E. coli K12 and S. aureus ATCC 29213, are tested independently. The bacteria are grown overnight at 37 degrees C. in trypticase soy broth (TSB, Difco Laboratories). The overnight cultures are diluted into fresh TSB and incubated at 37 degrees C. for 2.5 hours. The subcultures are adjusted to an optical density of approximately 0.2 in 10 mM aqueous sodium phosphate buffer at pH 7.4 (sodium phosphate, dibasic heptahydrate, NaPB, Mallinckrodt). These cell suspensions are further diluted 1:169 in 5.673% (w/v) TSB. These final dilutions of the bacteria are used as inoculum.

SLP1 and LP229 solutions at concentrations of 17 μM are serially diluted (two-fold, three replicates each) in NaPB. Each stock solution and dilution is added in duplicate (50 μL/well) to wells of a Costar 96-well flat bottom microtiter plate (Corning). NaPB is added in duplicate (50 μL/well) to the microtiter plate as a growth control. To each well containing SLP1, LP229, or NaPB (control), 10.6 μL of inoculum are added. The wells are slurried briefly (<20 seconds) and a 20 μL aliquot is removed from each well and processed to obtain data for the “Zero Time Point.” The 96-well microtiter plate is incubated with orbital shaking at 37 degrees C. for two hours. Another 20 μL aliquot is removed from each well and processed to obtain data for the “2-Hour Time Point.”

All samples from the “Zero Time Point” and “2-Hour Time Point” are processed similarly. Each sample is diluted into 20 μL of NaPB. Next, ten-fold serial dilutions are performed (20 μL into 180 μL of NaPB). Fifteen microliter aliquots of each serial dilution are spotted onto 5% sheep blood TSA II agar plates (BBL). The sheep blood agar plates are incubated overnight at 37 degrees C. The resulting colonies are counted to determine the number of Colony Forming Units (CFU)/mL. These data are used to determine the reduction in CFU of the test samples as compared to the control (as percent of control). Results, shown in Table 5, are plotted as concentration of LP229 or SLPI (μM) versus percent control as a measure of antibacterial activity. Results indicate that both SLP1 and LP229 exhibit antibacterial properties. For each protein, as the concentration of protein increases, bacterial CFU decrease.

TABLE 5
Effects of SLPI and LP229 on Growth of E. coli K12
and S. aureus ATCC 29213.

| | | Conc | % | | Conc | % |
|---|---|---|---|---|---|---|
| Bacteria | Sample | (μM) | control | Sample | (μM) | control |
| E. coli | SLPI | 14 | 49 | LP229 | 14 | 57 |
| E. coli | SLPI | 14 | 38 | LP229 | 14 | 55 |
| E. coli | SLPI | 7 | 73 | LP229 | 7 | 72 |
| E. coli | SLPI | 7 | 56 | LP229 | 7 | 87 |
| E. coli | SLPI | 3.5 | 81 | LP229 | 3.5 | 72 |
| E. coli | SLPI | 3.5 | 87 | LP229 | 3.5 | 95 |
| E. coli | SLPI | 1.75 | 106 | LP229 | 1.75 | 98 |
| E. coli | SLPI | 1.75 | 80 | LP229 | 1.75 | 95 |
| E. coli | SLPI | 0.875 | 105 | LP229 | 0.875 | 99 |
| E. coli | SLPI | 0.875 | 98 | LP229 | 0.875 | 104 |
| E. coli | SLPI | 0 | 100 | LP229 | 0 | 100 |
| E. coli | SLPI | 0 | 100 | LP229 | 0 | 100 |
| S. aureus | SLPI | 14 | 92 | LP229 | 14 | 73 |
| S. aureus | SLPI | 14 | 96 | LP229 | 14 | 68 |
| S. aureus | SLPI | 7 | 106 | LP229 | 7 | 76 |
| S. aureus | SLPI | 7 | 102 | LP229 | 7 | 72 |
| S. aureus | SLPI | 3.5 | 98 | LP229 | 3.5 | 89 |
| S. aureus | SLPI | 3.5 | 105 | LP229 | 3.5 | 85 |
| S. aureus | SLPI | 1.75 | 101 | LP229 | 1.75 | 100 |
| S. aureus | SLPI | 1.75 | 88 | LP229 | 1.75 | 93 |
| S. aureus | SLPI | 0.875 | 94 | LP229 | 0.875 | 92 |
| S. aureus | SLPI | 0.875 | 96 | LP229 | 0.875 | 90 |
| S. aureus | SLPI | 0 | 100 | LP229 | 0 | 100 |
| S. aureus | SLPI | 0 | 100 | LP229 | 0 | 100 |

We claim:

1. A method of preventing or treating a disease or pathological condition comprising administering to a patient in need thereof a pharmacologically effective amount of an LP229 polypeptide.

2. The method of claim 1 wherein the LP229 polypeptide comprises a polypeptide sequence as shown in SEQ ID NO:2 or any biologically active fragment or fusion thereof.

3. A method as in claim 1 or 2 wherein said disease or pathological condition is a poor healing or chronic wound.

4. A method as in claim 3 wherein the poor healing or chronic wound is selected from the group consisting of pressure ulcers, diabetic ulcers, venous stasis ulcers, burns, and wounds to tissue in the gastrointestinal tract.

5. A method as in claim 1 or 2 wherein said disease or pathological condition is selected from the group consisting of skin diseases, sepsis, inflammation, immunodeficiencies, autoimmune diseases, infectious diseases, allergic diseases, and malignancies.

6. A method as in claim 1 or 2 wherein said disease or condition is exacerbated by massive neutrophil infiltration.

7. A method as in claim 1 or 2 wherein the patient is a mammal.

8. A method as in claim 1 or 2 wherein the patient is a human.

9. A method as in claim 1 or 2 wherein the LP229 polypeptide is administered in a single dose.

10. A method as in claim 1 or 2 wherein the LP229 polypeptide is administered in multiple doses.

11. A method of preventing or treating a disease or pathological condition comprising administering to a patient in need thereof a pharmacologically effective amount of an LP229 epitope-recognizing antibody.

12. The method of claim 11 wherein said disease or pathological condition is a skin disease, sepsis, inflammation, an immunodeficiency, autoimmune disease, infectious disease, allergic disease, or malignancy.


SEQUENCE LISTING (7 sequences)

SEQ ID NO: 1 - DNA, 1055 bp, Homo sapiens
  CDS (90)..(461)
ctttcctctcctgactaagtttctctggcttccctgaggctgcaggtgttaatctggggggccctgggccctgagccggcagcagaaatatgaggacccagagccttctcctcctgggggccctcctggctgtggggagtcagctgcctgctgtctttggcaggaagaagggagagaaatcggggggctgcccgccagatgatgggccctgcctcctatcggtgcctgaccagtgcgtggaagacagccagtgtcccttgaccaggaagtgctgctacagagcttgcttccgccagtgtgtccccagggtctctgtgaagctgggcagctgcccagaggaccaactgcgctgcctcagccccatgaaccacctgtgttacaaggactcagactgctcgggcaaaaagcgatgctgccacagcgcctgcgggcgggattgccgggatcctgccagaggctaattctgatttaggatctgtggctctgcacctaagctggggaccaacggaaagagttcacgatgggaggcctggggccctgcccgctggacagcactatctctaccagcggtggttccagccttctgataatcactggcctgctgacacttccctgcaacccatccacccctggtttctcctcctgggagtcaaagtccatagcctgagctcggaggaaggcctctgtatcaccccagtactctgcaccactgccatacgagcttcccacccttcctaacgctttcacaccaatccgtacatgctgcttcctccaccaaaaatgcccaattcaggcagaccctgacctctccctcaggcagcccaaccatccagaatgaatattcttgcagagttttccaaacatcagtcattcacctctttcatgattttcaccatacctacaaaatagcaccatgataggttgcacgctgcctgtaccaccatttacttaatgttttctttaaatggctcacttttgtatataaataaattcatttcaaaaaaaaaaaaaaagggcggccgccgactagtgagctcgtcgacccgggaattaat

SEQ ID NO: 2 - protein, 123 aa, Homo sapiens [sequence omitted]

SEQ ID NO: 3 - protein, 131 aa, Mus musculus
  misc_feature (1)..(131): mouse SLPI
MKSCGLLPFTVLLALGILAPWTVEGGKNDAIKIGACPAKKPAQCLKLEKPQCRTDWECPGKQRCCQDACGSKCVNPVPIRKPVWRKPGRCVKTQARCMMLNPPNVCQRDGQCDGKYKCCEGICGKVCLPPM

SEQ ID NO: 4 - protein, 131 aa, Rattus norvegicus
  misc_feature (1)..(131): rat SLPI
MKSCGLFPLMVLLALGVLAPWTVEGGKNDAIKIGACPAKKPAQCLKLEKPECGTDWECPGKQRCCQDTCGFKCVNPVPIRGPVKKKPGRCVKFQGKCLMLNPPNKCQNDGQCDGKYKCCEGMCGKVCLPPV

SEQ ID NO: 5 - protein, 132 aa, Homo sapiens
  misc_feature (1)..(132): human SLPI
MKSSGLFPFLVLLALGTLAPWAVEGSGKSFKAGVCPPKKSAQCLRYKKPECQSDWQCPGKKRCCPDTCGIKCLDPVDTPNPTRRKPGKCPVTYGQCLMLNPPNFCEMDGQCKRDLKCCMGMCGKSCVSPVKA

SEQ ID NO: 6 - protein, 10 aa, Artificial
  Synthetic construct FLAG tag
DYKDDDDKHV

SEQ ID NO: 7 - protein, 20 aa, Artificial
  Synthetic construct FLIS tag
GAFIDYKDDDDKHVHHHHHH